(19) United States
(12) Patent Application Publication
(10) Pub. No.: US 2013/0193352 A1
(43) Pub. Date: Aug. 1, 2013

(54) METHOD FOR SETTING UP A RADIATION PLANNING AND METHOD FOR APPLYING A SPATIALLY RESOLVED RADIATION DOSE

(71) Applicants: Christoph Bert, Aschaffenburg (DE); Robert Luechtenborg, Darmstadt (DE)

(72) Inventors: Christoph Bert, Aschaffenburg (DE); Robert Luechtenborg, Darmstadt (DE)

(73) Assignee: GSI HELMHOLTZZENTRUM FUER SCHWERIONENFORSCHUNG GMBH, Darmstadt (DE)

(21) Appl. No.: 13/878,790

(22) PCT Filed: Oct. 7, 2011
(86) PCT No.: PCT/EP2011/067553

§ 371 Date: Apr. 11, 2013

(30) Foreign Application Priority Data
Oct. 12, 2010 (DE) 10 2010 048 233.1

(51) Int. Cl. A61N 5/10

(52) U.S. Cl. CPC A61N 5/1037
USPC 250/492.1

(57) ABSTRACT

A method for drawing up an irradiation plan for a radiation-generating device that includes a plurality of irradiation positions that, at least one of partially or at times, correlate with at least one basic parameter that is present at a point in time of the implementation of the irradiation plan, includes giving greater consideration to correlations with the at least one basic parameter that are expected with greater probability for at least some of the irradiation positions.

### CROSS REFERENCE TO RELATED APPLICATIONS

This application is a U.S. National Phase application under 35 U.S.C. §371 of International Application No. PCT/EP2011/067553, filed on Oct. 7, 2011, and claims benefit to German Patent Application No. DE 10 2010 048 233.1, filed on Oct. 12, 1010. The International Application was published in German on Apr. 19, 2012 as WO 2012/049085 A1 under PCT Article 21 (2).

### FIELD

The invention relates to a method for drawing up an irradiation plan for a radiation-generating device. Moreover, the invention relates to a method for administering a spatially resolved radiation dose in at least one irradiation position using at least one radiation-generating device. Furthermore, the invention relates to a device for drawing up an irradiation plan, to a device for actuating at least one radiation-generating device as well as to a radiation-generating device that comprises at least one device for actuating the radiation-generating device.

### BACKGROUND

Nowadays, objects are irradiated in all kinds of realms of technology. Depending on the concrete requirement of use, a wide array of very different irradiation methods as well as various types of radiation (for example, photon radiation, particle radiation, etc.) are used.

In many technical fields, for example, there is a need to irradiate objects two-dimensionally or three-dimensionally, whereby the radiation should act as uniformly as possible. This requirement is present, for example, if materials are to be hardened or changed in some other way in terms of their material properties. In the realm of food technology, it has now become quite common to impart food products with a longer shelf life by using certain types of radiation.

In other areas of technology, in contrast, it is only necessary to irradiate certain regions of the object to be irradiated with a specific, typically very high, dose while the other parts of the object are irradiated as little as possible or not at all. An example of this is the structuring of microprocessors or other microstructures or nanostructures using electromagnetic radiation (in some cases, all the way into the X-ray range and beyond) as well as imaging masks.

A locally varying dose distribution is not necessarily structured only two-dimensionally, but rather, in some areas of technology, it is also structured three-dimensionally. With such a three-dimensional structuring of the effective radiation, it is possible, for example, to directly and indirectly irradiate a volume area that is inside a body to be irradiated, without having to damage or open the body (especially its outer shell).

In this process, the problem often arises that the body to be irradiated (or a volume area located inside it) is not present only in a static or unmoving state. On the contrary, it can happen that the body or parts of thereof (especially the volume area to be irradiated) are moving. A movement can be made not only translatorily relative to an external coordinate system, but also in the form of a shift of various regions of the body that is to be irradiated relative to each other (including twisting, deformation, compression and/or stretching).

In order to be able to irradiate such (intrinsically) moving bodies, so-called 4D irradiation methods (four-dimensional irradiation methods) are employed. In actual fact, these are three-dimensional irradiation methods that have a temporal variation (with time as the fourth dimension). Examples of such material-processing methods can be found in the realm of material sciences in the production of highly integrated components (especially microprocessors and memory chips) as well as in the production of microstructured and nanostructured mechanisms.

Another field of technology that makes use of three-dimensional and four-dimensional irradiation methods is that of medical technology. Here, too, as a rule, it is necessary to irradiate specific volume areas inside a body, for instance, a tumor. The term “irradiation” refers to the exposure of the volume area inside the body to radiation, for example, particle radiation or photon radiation. In particular, it is necessary to expose the specific volume area to the highest possible dose. The surrounding tissue should only be exposed to a radiation dose to the smallest extent possible, or preferably essentially not at all. This is particularly the case when the surrounding tissue is a so-called critical tissue such as, for example, a sensitive organ (usually referred to in technical terminology as OAR, short for “organ at risk”). This can be, for instance, the spinal cord, blood vessels or nerve nodes.

Especially in the realm of medical technology, for obvious reasons, the employed methods should function precisely and error-free. Another problem in the realm of medical technology is that, due to different circumstances (e.g. the different body build of the patient, different position, different size, different characteristics of the tumor), there are actually always different starting conditions. This means that the radiation (treatment) of each patient has to be carried out on a case-to-case basis. As a rule, based on the current state of the art, these individual properties are taken into account during the irradiation planning, making use of so-called irradiation plans. Here, a dose distribution as prescribed by a physician for the various tissue regions is converted into “machine-readable parameters”. In other words, a set of parameters is computed for the device that generates the radiation, indicating the manner in which the patient has to be exposed to the radiation, for example, to the particle beam, in order to receive the best possible dose distribution prescribed by the physician. In this context, for example, the beam position, the beam energy, the beam incidence direction, the time management of the particle beam (scanning motion), the type of particle and the like are determined. In drawing up the irradiation plan, a large number of non-linear and complex interrelationships have to be considered. Thus, for example, in the case of charged particles, there is a need to take into account the dose deposition (actually undesired) into the tissue located behind the Bragg peak, but especially into the tissue in front of the Bragg peak. Furthermore, the so-called relative biological effectiveness (RBE) has to be computationally taken into account. The effect of the radiation on the tissue depends on different parameters such as the type of radiation, the type of particle, the particle energy and the type of irradiated tissue itself. Furthermore, effects due to secondary radiation have to be taken into account.

The already present complexity is even further increased if the patient to be irradiated or the target volume area to be irradiated moves. Special methods are needed in order to still be able to carry out a precise treatment. Currently, two special methods are often used to solve this problem, namely, so-called gating methods as well as so-called tracking methods. In the gating method, the irradiation plan is optimized in terms of a specific movement state of the patient. If the patient is in this particular movement phase, then a suitable radiation deposition is carried out. However, if the patient is in another movement phase, then the tissue of the patient would be irradiated “completely incorrectly” and erratically. In order to avoid such erroneous irradiation, which is generally unacceptable, no irradiation takes place whenever the patient is in a movement phase that falls outside of a certain movement window. It is easy to see that, as a rule, this significantly prolongs the duration of the irradiation procedure. This is not only unpleasant for the patient (since he/she has to undergo treatment for a longer period of time), but especially also leads to a marked increase in the costs, which is likewise undesired.

In the tracking method, the approach pursued involves a continuous tracking of that the movement of the patient. In this process, the movement of the patient is compensated for by a suitable repositioning of the particle beam. A repositioning in the transversal direction can be carried out by means of deflecting magnets. A repositioning in the longitudinal direction can be carried out, for example, by means of energy modulators. Such energy modulators consist, for example, of a double wedge pair, whereby the relative position of the individual wedges of the double wedge pair to each other as well as to the particle beam is variable. Depending on the position of the wedges, the particle beam that passes through the double wedge pair traverses a different path through the (energy-absorbing) material of the wedges. The energy of the particles is damped accordingly. In this manner, the energy of the particles and thus the position of the Bragg peak, can be set within certain limits. In the case of tracking methods, the irradiation planning, which is still necessary, is carried out in that an irradiation plan is drawn up for a specific reference movement phase. The deviations between the current movement phase and the reference movement phase are compensated for by the described repositioning of the particle beam. The parameters required for the repositioning of the particle beam are normally part of the optimization of the irradiation plan. Using such tracking methods, the dose that is supposed to be administered in the current target raster voxel can actually be administered there (in spite of a possible change in the position). One problem, however, is the surrounding tissue (especially the tissue located proximally in the beam direction (“upwind-side”) relative to the target raster voxel). For example, if tissue is compressed or stretched due to a movement, it normally happens that a biologically effective dose that deviates markedly from the reference plan is deposited in those particular regions. If the tissue to be irradiated is also rotating, then it can also occur that the particle beam will run through different tissue regions than is the case in the reference plan, as a result of which a dose is deposited in regions that are not provided for in the reference plan (and vice versa). The actually deposited dose (also outside of the target raster voxel) can be measured and computed during the actual irradiation, but this actual dose deposition is not predictable because of the correlation between the movement phase and the actually irradiated target raster voxel, which is unknown before the irradiation. As a rule, at best, approximated predictions can be made using statistical models. Thus, however, it is not possible to (exactly) predict the cumulative dose. Consequently, the resultant irradiation plan has deficits in this realm. This can result in an impairment of the irradiation quality, especially due to potential under-dosing in the tumor region and/or due to potential overdosing in the surrounding tissue.

Thus, there continues to be a need for improved irradiation methods and/or improved computation methods. In particular, the methods should become more precise and/or more cost-effective.

### SUMMARY

In an embodiment, the present invention provides a method for drawing up an irradiation plan for a radiation-generating device that includes a plurality of irradiation positions that, at least one of partially or at times, correlate with at least one basic parameter that can be present at a point in time of the implementation of the irradiation plan. The method includes giving greater consideration to correlations with the at least one basic parameter that are expected with greater probability for at least some of the irradiation positions.

### BRIEF DESCRIPTION OF THE DRAWINGS

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1 shows a preceding procedure for drawing up an irradiation plan in the form of a schematic flow diagram;

FIG. 2 shows a procedure for implementing an irradiation plan for the irradiation of a moving target volume, in a schematic flow diagram;

FIG. 3 shows a device for carrying out an irradiation, in a schematic perspective view;

FIG. 4 shows an illustration of a method for adapting the irradiation speed by varying the pause times; and

FIG. 5 shows an illustration of a method for adapting the irradiation speed by varying the radiation rate.

### DETAILED DESCRIPTION

In an embodiment the present invention provides a method that has been improved in comparison to the state of the art for drawing up an irradiation plan for a radiation-generating device, a method that has been improved in comparison to the state of the art for administering a spatially resolved radiation dose in at least one irradiation position using at least one radiation-generating device, an improved device for generating an irradiation plan, an improved device for actuating at least one radiation-generating device and/or an improved radiation-generating device that comprises at least one device for actuating the radiation-generating device.

A method for drawing up an irradiation plan for a radiation-generating device is proposed in which the irradiation plan comprises a plurality of irradiation positions that, at least partially and/or at least at times, correlate with at least one basic parameter that can be present at the point in time of the implementation of the irradiation plan, said method being carried out in such a way that, when the irradiation plan is being drawn up, greater consideration is given to correlations with the at least one basic parameter that can be expected with greater probability for at least some of the irradiation positions. Drawing up an irradiation plan is a numerically very complex process. For this reason, it is advantageous to limit this procedure to the “essential parts” when computing the irradiation plan. The essential parts here are especially the ones that occur relatively frequently, for example, from a statistical standpoint, where pronounced fluctuations occur (for instance, due to non-linear effects, especially tissue boundaries—for example, from a bone region to a “normal tissue region”—and the like). If one accordingly handles such especially relevant regions (particularly in comparison to other regions) with a higher precision (for example, with a finer computational grid, a higher numerical precision, improved computation methods and the like), then the overall precision of the irradiation plan can be improved, without a (substantial) increase in the numerical work needed for drawing up the plan. In certain cases, it is even possible to achieve a reduction in the work involved in drawing up the irradiation plan, while maintaining (essentially) the same or even an improved quality of the irradiation plan. The “greater consideration” can be given here over a broad scope. Thus, for instance, it is possible for the computation to involve a greater precision of the computational grid and/or a greater precision of the algorithms and/or a greater number of iteration steps. In the extreme case, a “binary” (or “quasi-binary”) emphasis of the appertaining correlation can also be an option. In such a case, a computation (establishing actuation parameters, dose deposition in tissue areas that deviate from the current target raster voxel, and the like) is carried out only for the correlation that is probable or expected or most likely to occur (“binary computation”), or only for the correlation (preferably including the correlation that is probable or expected or most likely) that is in the vicinity of the correlations that are probable or expected or most likely (“quasi-binary computation”). If the basic parameter is, for example, a movement cycle (for instance, a breathing cycle), the various irradiation positions can each be correlated with a (preferably “suitable”) movement phase of the movement sequence. It is conceivable that a radiation is only permitted at the points in time when the appertaining correlation does indeed (approximately) occur (that is to say, a kind of “gating” method is carried out). In contrast to previous “traditional” gating methods, however, the efficiency of the resulting method can at times be markedly improved by the proposed greater consideration of correlations that can be expected with greater probability. Moreover, it is at times especially possible to markedly improve the precision vis-à-vis “classic” tracking methods. This relates particularly to volume areas that fall outside of the currently irradiated target point voxel. The improvement becomes especially clear when compressive and stretching effects and/or tissue rotations play a greater role. The plurality of irradiation positions, each having one (optionally also several) correlating basic parameter, can also be seen as a kind of gating method with a plurality of gating time windows. Here, for example, each movement phase (in case of a moving body to be irradiated) can be assigned its own gating window. Altogether, the individual gating windows can add up, so that ultimately, a relatively large percentage of the total irradiation time can bring about a given radiation deposition into the body to be irradiated. Preliminary experiments have shown that 20%, 30%, 40%, 50%, 60%, 70%, 80% or 90% (and more) of the irradiation time can be used effectively. Moreover, it is possible for the irradiation plan to be computed in such a way that, within a single gating window or if a certain basic parameter value occurs or if a basic parameter interval occurs, a (“classic”) tracking method is provided for purposes of further improving the irradiation plan, and accordingly, such device parameters are computed and stored. However, preliminary experiments have shown that (especially with a large number of basic parameters or basic parameter intervals or gating windows), it is often possible to dispense with such tracking (particularly if “regular” operating conditions are present), since the inaccuracies that can be induced by the tracking can at times even worsen the accuracies that can be achieved with the proposed method (or the proposed irradiation plan). In addition to the already mentioned advantages (especially the greater precision and/or the reduced numerical work), another advantage of the proposed method is a reduction of the data quantities that have to be stored or processed. Moreover, it is advantageous if, in drawing up the irradiation plan, it is taken into consideration that, during the irradiation that is ultimately carried out using the irradiation plan, the irradiation sequence can be varied in such a way that the actually occurring basic parameter(s) and the basic parameter(s) which, when the irradiation plan was drawn up, is/are assumed to correlate with a specific irradiation position, correspond/can correspond with each other, at least essentially, and/or are adapted/can be adapted.

For the rest, it is also possible (and as a rule also advantageous) that, already when the irradiation plan is being drawn up, certain basic parameter ranges of at least one basic parameter and/or certain correlations between at least one basic parameter and at least one irradiation position can be excluded at least at times and/or at least in certain areas. This exclusion can especially be effectuated in that, at a later point in time, especially within the scope of implementing the irradiation plan, these basic parameter ranges or such correlations do not occur or are not implemented. For example, it is possible that certain breathing phases (for example, those during which a higher dose deposition is to be expected into organs at risk) are completely or partially (for example, for certain energy layers) excluded from the irradiation plan. This can take place in that, in such a case, the particle beam is “paused” and/or is moved to the next “meaningful” irradiation volume.

It is advantageous if, with the methods, a division into individual irradiation positions and/or a division into groups of irradiation positions is made, at least at times and/or at least partially time-based and/or at least at times and/or at least partially as a function of the basic parameters. As a rule, a time-based division can be implemented especially simply and quickly. In contrast, with a basic parameter-dependent division, any previous knowledge that might be available can be meaningfully taken into consideration. An example would be if, in case of a cyclic movement of a target volume area that is to be irradiated, the division into irradiation positions or into groups of irradiation positions is made as a function of the movement of the target volume area, especially as a function of a movement phase of the target volume area. For example, irradiation positions or groups of irradiation positions that correlate with movement phases during which the target volume area is only moving slowly (for example, in the inhaled or exhaled state) can be selected to be relatively large (especially in terms of the time window they cover). At points in time in which the target volume area is moving quickly (for example, during the inhalation or during the exhalation), in contrast, the irradiation positions or the groups of irradiation positions can be relatively small. This “being large” or “being small” can relate especially to the time axis, so that, particularly in the described example, the spatial resolution can remain essentially constant.

It can also be advantageous if a time-variable dose rate of the radiation generated by at least one radiation-generating device is at least at times and/or at least partially taken into consideration when the irradiation plan is being drawn up, and/or if a safety margin is taken into consideration when the irradiation plan is being drawn up. It has been found that, especially with particle accelerators that operate non-continuously or that operate intermittently, the number of particles does not necessarily remain constant (in a synchrotron, for instance, the time-dependent course of the so-called particle spill). The form of the particle spill, however, can remain relatively constant from one particle spill to the next. If this is taken into consideration during the irradiation planning, the quality of the irradiation that is ultimately carried out might even be further improved. Particularly if a safety margin is taken into account when the irradiation plan is being drawn up, then fluctuations—which can never be completely avoided in actual practice—between the assumed basic parameter sequence and the actual basic parameter sequence can, as a rule, be very advantageously compensated for during the actual irradiation. As a result, it is at times possible to significantly improve the quality of the irradiation that is ultimately carried out.

Although it is possible to take the basic parameters into consideration on the basis of statistical or other deliberations when the irradiation plan is being drawn up, as a rule, it is practical if the method is carried out in such a way that the at least one basic parameter is incorporated into the irradiation plan at least at times and/or at least partially while making use of data preferably obtained by measurements. For example, it is possible to examine a patient making use of diagnostics (for example, imaging diagnostics such as computer tomography methods and/or nuclear magnetic resonance tomography methods) before the irradiation plan is drawn up. In this process, for example, data can also be obtained (4D acquisition) about a movement pattern (for example, a typical breathing cycle) that is typical of the patient in question. This data is appropriately incorporated into the drawing up of the irradiation plan. In particular, the time-related behavior obtained here can be used to achieve an especially advantageous correlation between movement phases and irradiation points. If an appropriate safety margin is taken into account here, then the irradiation that is ultimately carried out can typically be adapted in such a way that a faster or slower movement sequence can be compensated for during the later irradiation.

It is especially advantageous when the method is carried out in such a way that the irradiation plan is drawn up in several steps, at least at times and/or at least in certain areas. In particular, it is possible to carry out at least one drawing-up step of the irradiation plan and/or at least one drawing-up step of at least one partial irradiation plan for at least one irradiation position and/or at least one optimization step of the irradiation plan and/or at least one optimization step of at least one partial irradiation plan for at least one irradiation position. In this manner, as a rule, it is possible to save computing time (especially at certain critical points in time) without the quality of the irradiation planning having to drop (excessively) in this process. It has been found that the initial drawing up of the “initial irradiation plan” is particularly demanding in terms of computation. This step should preferably only be carried out a single time. As a rule, however, slight to moderate deviations from the originally drawn-up “initial irradiation plan” can be made employing optimization methods. However, the optimization is numerically much less demanding (for example, because fewer iteration steps have to be carried out), so that computing time can be saved here. If applicable, a subsequent optimization can be made immediately before the irradiation, after the patient has been immobilized if data is already available about the “current-day behavior” of the radiation-generating device and/or of the actual basic parameter sequence (for example, a current movement sequence such as especially the current breathing movement of a patient and the like on that given day).

In addition or as an alternative, it is proposed to carry out a method for administering a spatially resolved radiation dose in at least one irradiation position using at least one radiation-generating device in such a way that the dose rate deposited by the at least one radiation-generating device in the at least one irradiation position is varied at least at times and/or at least partially in correlation with at least one basic parameter that is present at the time of the administration. As the radiation-generating device, it is preferable to use a beam-generating device, especially preferably a particle beam-generating device, since, as a rule, (charged) particles have a more or less pronounced Bragg peak, so that a “movement” of the particle beam is also possible in the longitudinal direction. Moreover, it is also possible for radiation-generating devices to have a temporally fluctuating output intensity. Furthermore, it has been found in actual practice that certain variations practically always occur in a biological system. Even though, for example, the breathing cycle in the same individual is often relatively similar from one cycle to the next, it can nevertheless happen that the breathing cycle can be shortened or lengthened, for instance, due to temperature effects, nervousness and so on. The result of both of these effects is that, when scanning methods are used, a coincidental correlation can occur between the irradiation position (position of the target volume that is to be irradiated) and the movement phase during which the dose is deposited in this irradiation position. Consequently, as already mentioned, it is still possible that—in spite of the movement—the dose is indeed deposited in the irradiation position that is actually to be irradiated. However, the undesired but unavoidable dose depositions into the surrounding tissue (especially in the particle beam direction, into tissue regions that are situated proximally to the irradiation position) cannot be (precisely) predicted. Until now, this has been viewed as an intrinsic problem that cannot be solved. In particular, a number of previously conducted experiments that have suggested adapting the breathing of the patient to the properties of the beam-generating device have failed. This is particularly the case when “soft” methods are used (for example, requesting the patient to breathe in a certain manner). In this context, some “rigid” methods have already been carried out such as ventilation under anesthesia, but these are quite laborious as well as burdensome to the patient, as a result of which they are subject to criticism. Therefore, the inventors are proposing that, when the irradiation is performed, the adaptation to (at times varying) basic parameters that occur during the course of the irradiation (such as the breathing of a patient) should take place in such a way that the radiation-generating device (especially its dose rate or the length of irradiation pauses) is readjusted. This makes it possible for the “nominal” relationship between the movement phase and the irradiation position to be retained (at least as a reasonable approximation), even when certain fluctuations in the basic parameter occurs (such as, for instance, the movement). Since the “nominal” relationship between the irradiation position and the basic parameter is retained (that is to say, their correlation), it is now also possible for the irradiation planning to “predictively” take into account, for instance, dose depositions outside of the current irradiation position. In this manner, the precision of the irradiation can often be markedly improved.

It is especially advantageous if the method for administering a spatially resolved radiation dose is carried out in such a way that the administration of the spatially resolved radiation dose is performed at least at times and/or at least partially and/or at least in certain areas while making use of at least one irradiation plan. In particular, an irradiation plan of the above-mentioned type can be used in this context. Precisely when the drawing up of the irradiation plan involved making advantageous assumptions (for instance, determined by preceding measurements) regarding the basic parameters that occur during the actual irradiation (including their course over time) and when suitable safety margins were preferably taken into account, an especially precise irradiation can be carried out, in which especially also dose depositions in volume areas situated outside of the actual irradiation position—and thus the ultimately resulting cumulative dose distribution—can be predicted very precisely.

In particular, it is proposed for the method to be carried out in such a way that the at least one basic parameter represents a movement of at least one irradiation position, especially a periodic movement of at least one irradiation position that occurs at least at times and/or at least partially, and/or represents a dose rate of the at least one beam-generating device, especially a time-varying maximum dose rate of the at least one radiation-generating device, and/or represents a time variation of the dose rate generated by the radiation-generating device. Preliminary experiments have shown that the proposed methods (individually as well as in combination) are especially effective when the basic parameter represents a movement of at least one irradiation position and/or for a dose rate of the radiation-generating device. If at least one irradiation position (or a target volume area) moves, then it is especially advantageous (particularly in terms of a reduction of the number of computations that must be performed) if the movement takes place at least partially cyclically and/or periodically. Here, the total cycles or total periods can especially advantageously be divided into individual partial sections. In case of fluctuations of the dose rate of the at least one radiation-generating device, this can especially be a time-varying maximum dose rate of the at least one radiation-generating device. This can be, for instance, the number of particles released per unit of time during a so-called particle spill in a particle synchrotron.

It is especially advantageous if the method/methods is/are used when the movement of at least one irradiation position, at least at times and/or at least in certain areas, is a translatory movement, a rotatory movement, or movements that shift relative to each other, a compressive movement and/or a stretching movement, and/or a change in terms of density. It is precisely with such movements that the methods known until now often entail major problems so that, thanks to the present invention, particularly clear improvements are possible.

Moreover, it can be advantageous if the at least one radiation-generating device generates particle radiation at least at times and/or at least partially, especially hadron radiation at least at times and/or at least partially, preferably proton radiation at least at times and/or at least partially, helium ion radiation, carbon ion radiation, neon ion radiation, oxygen ion radiation, pion radiation, meson radiation and/or heavy ion radiation. Particle radiation, especially the above-mentioned types of particle radiation, has already proven to be especially effective in the treatment of tumors in the past. This is especially due to the very pronounced Bragg peak of particle radiation, especially of the above-mentioned particle radiation. However, otherwise as well, the proposed particle radiation generally has a highly destructive effect on tumor cells, which is advantageous.

It is also advantageous if the deposited dose rate is varied at least at times and/or at least partially by varying the dose rate generated by the radiation-generating device, and/or by at least at times and/or at least partially discontinuing the dose deposition. The proposed methods for adapting the dose rate have proven to the especially effective or relatively easy to implement.

It is advantageous if, with the method, a smaller dose rate than the maximum possible dose rate is deposited under standard conditions. Generally speaking, an adaptation of the dose rate can be technically achieved relatively easily through the “removal” of particles to a greater or lesser extent or by withholding the administration of particles to a greater or lesser extent. (For instance, the application of the beam can be briefly discontinued.) With the proposed refinement of the method, thanks to the buffer that is now present, it is also possible to utilize the buffer to carry out a variation in the direction of a “higher dose rate”. This can once again increase the flexibility and/or the precision of the method. Of course, the magnitude of the safety margin should not be selected too large, since it prolongs the duration of the treatment proportionally to its magnitude. In preliminary experiments, a reduction of 10% to 20% in comparison to the maximum dose rate has proven to be a favorable value.

It is also being proposed that a correlation be established between at least one irradiation position and at least one basic parameter, especially a correlation between at least one irradiation position and at least one movement phase of the at least one irradiation position. Experiments have shown that especially due to such a correlation, particularly large, otherwise frequently occurring, errors can be eliminated or at least markedly reduced.

It is also especially advantageous if the irradiation takes place in the form of a scanning method, especially a raster scanning method. This has proven to be especially advantageous in conjunction with the proposed method(s). However, in particular, it is also possible to use so-called spot scanning methods as well as continuous scanning methods.

Furthermore, it is possible to provide a sort of temporary emergency switch-off within the scope of the method for administering a radiation dose. If, for example, the patient makes an “irregular” movement, he/she can then be protected from erroneous irradiation. For instance, during the treatment for a lung tumor of a patient, the breathing (for example, the movement of the chest or the like) can be monitored. If the patient coughs, the particle beam can be (briefly) switched off. This is advantageous, especially since it is normally the case that no appropriate irradiation plan can be drawn up for such irregular movements (for example, particularly fast movements). Moreover, when it comes to irregular movements, it is practically impossible to establish a meaningful correlation between the basic parameter and the irradiation position. After such a brief irradiation interruption, as a rule, it is appropriate to pause the irradiation until the current breathing phase is the same as the one that had been reached before the interruption. In addition or as an alternative, such a temporary irradiation interruption can also be employed if at least one of the basic parameters (for example, the breathing movement of a patient) reaches values that are not provided for in the irradiation plan. Such a case can occur if such values did not occur during a measurement that took place during the drawing up of the irradiation plan. Thus, for example, it is not uncommon during the actual treatment for a patient, at least at times, to breathe at a greater amplitude (“especially deep breaths”) than he/she did during the preliminary examination when the irradiation plan was being drawn up. In the opposite case, in which the breathing trajectory does not even reach certain movement phases (or if some other basic parameter does not reach certain values), if applicable, a sort of “emergency tracking” can be employed for these phases. The irradiation during these phases can then be carried out, for example, while using suitable tracking parameters during a preferably adjacent movement phase (basic parameter phase).

Moreover, a device for drawing up an irradiation plan is being proposed that is configured and equipped in such a way that it executes a method according to the above-mentioned description. The device for drawing up the irradiation plan then analogously has the already described advantages and properties.

Furthermore, a device for actuating at least one radiation-generating device and/or a radiation-generating device that comprises at least one device for actuating the radiation-generating device is being proposed which is configured and equipped in such a way that the resulting radiation-generating device, at least at times and/or at least partially, executes a method of the above-mentioned type. The device for actuating at least one radiation-generating device and/or the radiation-generating device then analogously have the already described advantages and properties.

Although so far, the elaborations have been focused essentially on the treatment of a (human) patient, it is, of course, also possible to use these proposals for animals, biological specimens (for example, cell cultures), patient dummies, phantoms and/or mechanical workpieces.

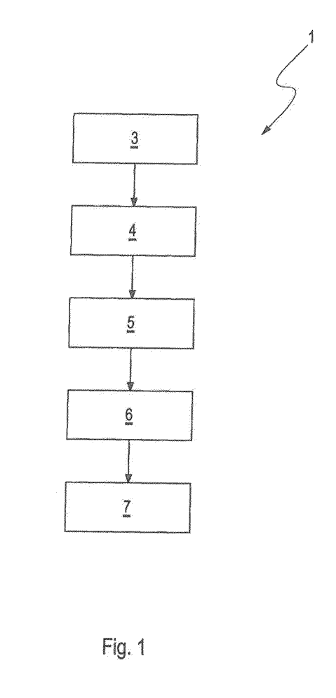

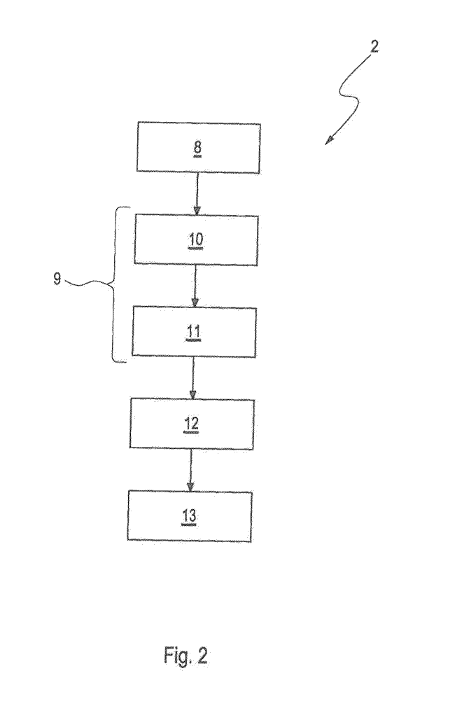

FIG. 1 shows a schematic view of a preceding procedure 1 for drawing up an irradiation plan. The preceding procedure 1 is typically carried out before the actual treatment (the actual irradiation procedure 2), and often at a different location. Typically, the preceding procedure 1 is carried out several days before the actual irradiation procedure 2 (see FIG. 2).

During the preceding procedure 1, first of all, the precise position and size of the tissue to be treated are determined in an examination step 3. Within the scope of the examination step 3, for example, imaging methods are used, especially computer tomography methods and/or nuclear magnetic resonance tomography methods. Furthermore, in the embodiment shown here, the planning data is acquired in a time resolved manner (that is to say, for example, by means of a 4D computer tomography method or 4D nuclear magnetic resonance tomography method). Moreover, it is also advantageous if data for a movement substitute is acquired at the same time (such as, for instance, for a strain gauge that is placed around the chest of a patient 22).

The data thus acquired is converted in a digitalization step 4 into a data format that is suitable for drawing up the irradiation plan numerically. This can be, for example, the digitalization of analog data. Even if digital data is already present, computations—at times complex ones—might be necessary for the conversion into a data format that is suitable for drawing up the irradiation plan. For example, in order to draw up an irradiation plan, it might be necessary or appropriate to determine tissue boundaries. Numerical methods can be used for this purpose, but in addition or as an alternative, there might also be a need for input by a person (if applicable, also interactively).

Parallel to this (not shown in FIG. 1), the examination result obtained in method step 3 can also be used by a physician to prescribe the dose that is to be administered during the actual irradiation procedure 2 (see FIG. 2).

The data acquired during the digitalization step 4—while taking into consideration the dose prescribed by the physician—is used in the next method step 5 in order to draw up a preliminary irradiation plan (initial irradiation plan). The initial irradiation plan in step 5 is drawn up, for example, using generally known methods. For instance, a “classic” tracking irradiation plan can be drawn up assuming a reference position.

The next method step 6 generates a set of “partial irradiation plans”—based on the initial irradiation plan set up in step 5. The type of subdivision as well as the number of “partial irradiation plans” are based here on the boundary conditions that are present in a concrete case. In this context, the individual “partial irradiation plans” refer to a certain number of irradiation positions 33 (if applicable, also to an individual irradiation position 33). It is especially advantageous if, during the division of the initial irradiation into several partial irradiation plans, measured values that were obtained in examination step 3 are taken into consideration. For example, if a tumor in the lung region of a patient 22 is to be irradiated, then especially the breathing movement 29 of the patient has to be taken into consideration in drawing up the set of partial irradiation plans. Advantageously, in case of a moving tumor, the “division” of the initial irradiation plan into partial irradiation plans is carried out while taking into consideration the movement pattern of the tumor or of the surrounding tissue. Thus, after a movement of the tumor by, for example, 2 mm, a new partial irradiation plan can be provided. As a result, the specific speed of the tumor tissue can be incorporated indirectly into the drawing up of the partial irradiation plan. As a rule, the partial irradiation plans are thus not at equal time intervals. Merely by way of example, a partial irradiation plan that correlates with a fully inhaled state of the patient 22 (low tumor speed) relates to a larger number of irradiation positions than a partial irradiation plan that correlates with a movement state 30 of the patient 22 in which the patient is currently inhaling (high tumor speed).

However, under certain boundary conditions, it might also be advantageous for the partial irradiation plans to be (at least at times) at equal time intervals.

It is preferable if the irradiation speed of the irradiation system 14 is incorporated into the drawing up of the partial irradiation plans (and thus into the final irradiation plan). This can be adequately familiar or—if it fluctuates—can be measured in close proximity to the time of the actual treatment.

Moreover, the time sequence of the individual partial irradiation plans is configured here in such a way that the total irradiation plan drawn up on the basis of the individual partial irradiation plans can be carried out in the previously computed form, especially at a dose rate that is reduced in comparison to the maximum dose rate of the irradiation system 14. A typical value is one in which the irradiation plan can be carried out at a dose rate that is reduced by 10% to 20%. Thanks to this “safety supplement”, it is possible to compensate, on the one hand, for a faster breathing movement 29 of the patient 22 (caused, for instance, by nervousness) as well as for a time-varying dose rate of the irradiation system 14 itself. Of course, an irradiation plan provided with such a “safety supplement” can also be carried out if the irradiation system has a higher dose rate (for example, pause intervals 32 during which the beam is not applied can be inserted in appropriate quantities and/or with a suitable interval length). Through this measure, the correlation between the movement phase 30 of the tumor and the irradiation position in question “assumed” in the irradiation plan can also be maintained during the irradiation 2 that is to be carried out later.

For each computation of the partial irradiation plans in step 6, depending on the actual requirements, it is possible to use optimization algorithms (based on the initial irradiation plan drawn up in step 5). With such an approach, normally a marked reduction in the numerical work can be achieved, without much loss of quality. However, under certain boundary conditions, it has also proven to be advantageous to compute an entirely new partial irradiation plan (for example, making use of the method employed in step 5 for drawing up the initial irradiation plan). However, the additional time thus needed increases the effort. However, as a rule, this greater time requirement is irrelevant for the patient since there are usually several days between the preceding procedure 1 and the actual irradiation procedure 2 anyway, and thus there is sufficient time available to carry out even extensive computations.

In the optimization step 6, the adaptation parameters of the tracking plan (for example, transversal and/or longitudinal shift of the particle beam) can be taken into consideration entirely, only partially or even not at all. An advantage of not taking, for example, the deep-layer modulation (longitudinal shift of the particle beam) into consideration can be, for instance, that no technically complex adaptation of the particle energy is necessary during the irradiation. Owing to the known correlation between the irradiation point (actually irradiated target volume) and the movement phase, the quality of the ultimately employed dose distribution can nevertheless be sufficiently high.

Only for the sake of completeness, it should be pointed out that, when suitable algorithms are used, a final irradiation plan can also be drawn up directly (without method step 6 involving additional optimization algorithms being carried out).

Finally, the acquired data (that is to say, especially the initial irradiation plan as well as the individual partial irradiation plans) are stored 7. Any desired storage media, such as hard drives, CDs, DVDs, blue-ray discs, solid-state memory devices (for example, USB sticks) and the like, can be used for this purpose. Incidentally, commercially available computers (PCs, workstations and the like) can be used in order to draw up the irradiation plan itself. Of course, it is also possible to use specialized hardware for drawing up at least parts of the irradiation plan.

FIG. 2—likewise in the form of a schematic flow diagram—shows the actual irradiation procedure 2.

At the beginning of the irradiation procedure 2, the preparatory measures 8 are carried out first. Thus, the patient 22 is immobilized at the actual treatment site 21 and the irradiation plan computed during the preceding procedure 1 is read into the irradiation system 14. Moreover, in the embodiment shown here, for the further execution of the method, the current movement behavior of the target volume that is to be irradiated (for example, the breathing movement 29 in the case of the treatment of a tumor in the lung region) and the extraction behavior of the accelerator 14 (irradiation speed) are once again calibrated. This measure makes it possible to determine the status of the tumor movement that is current for that particular day or for the treatment cycle, on the one hand, and the status of the irradiation system 14 (especially of the heavy ion accelerator 15) on the other hand. Particularly these two influencing variables essentially determine the correlation between a given irradiation point 33 and the movement phase 30. In this context, one should think specifically in terms of a possible time-variable dose rate over an individual particle spill. This step could also be dispensed with, especially in case of a heavy ion accelerator 15 whose behavior is sufficiently precise or reproducible, and in case of a patient 22 whose tumor trajectory 29 is adequately reproducible. In order to increase the precision, it is also optionally possible to shoot the prepared irradiation plan computed during the preceding procedure 1 into an empty CAVE (or into a patient dummy and/or into a phantom) for test purposes. Another optional method step is to once again perform a complete time-resolved measurement of the patient 22 (for example, in the form of a four-dimensional computer tomogram and the like). Here, optionally, the precision of the subsequent treatment can be further increased. In view of the extra work this entails, however, such a repeated complete measurement is usually limited to exceptional cases.

Now, in the embodiment of the irradiation procedure 2 being presented here, a re-optimization of the irradiation plan drawn up during the preceding procedure 1 is carried out in the subsequent method step 9. This re-optimization 9 is carried out in the form of several sub-steps that are described in greater detail in FIG. 2. The decision as to whether a re-optimization 9 is to be carried out or not can especially be made as a function of the basic parameters present (such as particularly the anatomy of the patient 22, the current breathing 29 of the patient 22 and/or the current accelerator status).

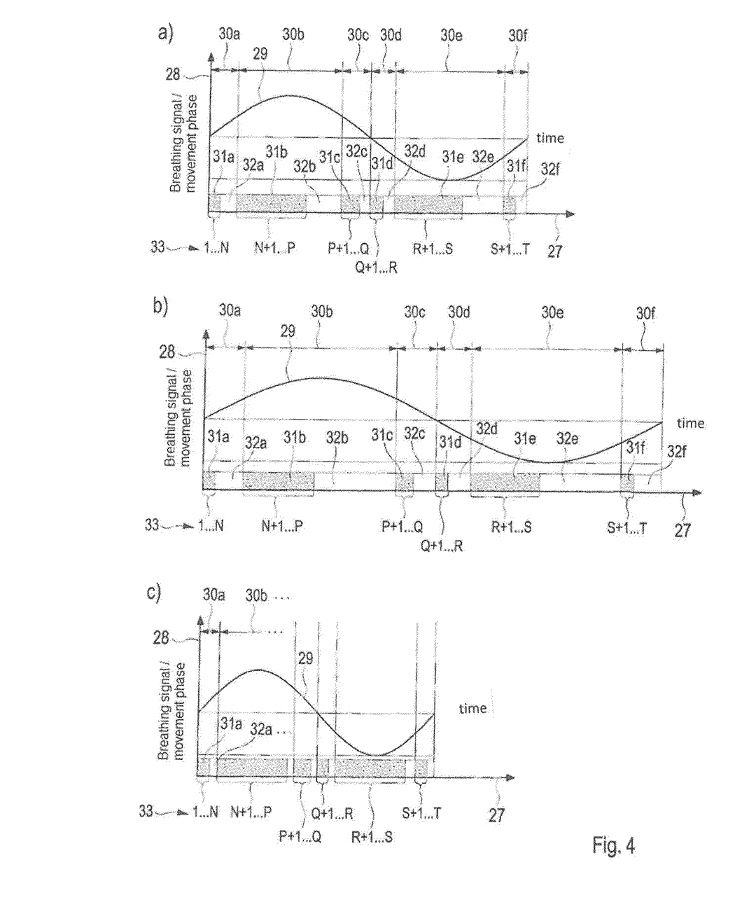

First of all, current time intervals 31, 32 for the movement phase sequence 30 for that particular day are determined 10 on the basis of the data and measured results obtained during the preparatory measures 8 (especially in terms of the current movement pattern 29 of the patient 22 as well as the current behavior of the heavy ion accelerator 15 or other parts of the irradiation system 14 on that particular day). Here, too, the re-optimization 9 of the irradiation plan as well as the division 10 into individual movement phases 30 are carried out in such a way that the irradiation plan that is to be subsequently re-optimized 9 can be implemented especially at a reduced dose rate of the irradiation system 14. In the irradiation procedure 2 being presented here, the irradiation speed is adapted by incorporating pause phases 32 (see FIG. 4) into the irradiation plan, especially at least one pause phase 32 per movement interval 30. This can be done, for instance, in such a way that the points 33 that are to be irradiated during a single irradiation phase 30i over the time span t_{B,i} can be irradiated during a time span t_{B,i}-t_{safety} that has been reduced by a safety margin t_{safety}. During the actual irradiation, by varying the pause phase 32 t_{safety}, the irradiation can be carried out in such a way that, even in case of the occurrence of fluctuations, the raster points 33 that are to be irradiated during the moment phase 30i can be irradiated. Consequently, as a rule, the time span t_{B,i} of the individual movement phase 30i deviates from the time span t_{B,i} assumed in the irradiation plan. The correlation (assumed in the irradiation plan) between the raster points 33 and the movement phase 30, however, can be maintained (in actual practice, generally with a good to excellent approximation).

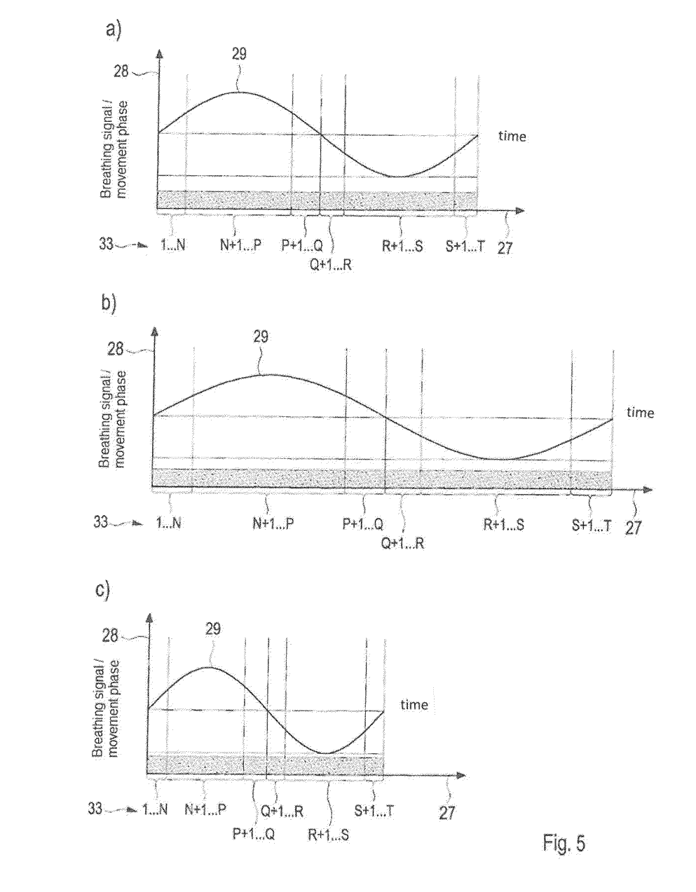

In addition or as an alternative, in order to adapt the irradiation speed, the plan can work with a modified maximum dose rate (which is below the maximum dose rate of the irradiation system 14), if an irradiation system 14 is used in which the dose rate can be adapted during the treatment in real time (as sufficiently fast) to the changes in the tumor movement (see FIG. 5).

After method step 10, it is clear which raster point 33 is irradiated during which corresponding movement phase 30. Moreover, it is ensured that only one single movement phase 30 is present (when the pause phase adaptation is used) per single time interval (gating window; length t_{B,i}-t_{safety}).

The following step is the drawing up of an updated irradiation plan (step 11), based on the four-dimensional irradiation plan (set of individual partial irradiation plans) determined during the preceding procedure 1. The updated irradiation plan can be drawn up in a first step by re-sorting the data already acquired during the preceding procedure 1 and during an optimization step subsequently carried out for the irradiation plan that has been re-sorted in this manner. The re-optimization 9 of the updated irradiation plan is carried out here with the inclusion of the non-linear effects such as, for example, the non-linear influence of the biological effect (RBE=relative biological effectiveness). Step 11 for drawing up the updated irradiation plan is numerically demanding, but its scope is still limited to such an extent that it is possible to carry out the re-optimization procedure 9 in immobilized patients 22. This is especially possible since, within the scope of the preceding procedure 1, an initial database has already been created whose optimization during the re-optimization procedure 9 is relatively less complex than drawing it up the first time during the preceding procedure 1.

In order to further increase the irradiation safety, in a test irradiation step (not drawn separately here), the irradiation plan that was re-optimized in step 9 can be implemented for a dummy, on the basis of the real patient data (including the currently measured body movement) as well as on the basis of the particle accelerator data.

The actual therapeutic irradiation 12 or treatment that is now administered is carried out, so to speak, as a gated irradiation, whereby, however, it is not a single gating window that is used for the entire movement cycle (as is the case with “classic” gating methods), but rather a plurality of gating windows 31a, 31b, . . . are used, which each apply to different movement phases 30 of the patient 22 (incidentally, a test irradiation that might be carried out should preferably also be administered as an irradiation that is gated multiple times). As described above, through the adaptation of the length of the gating pauses 32, it is ensured that the correlation between the raster point 33 and the movement phase 30 will be obtained. Consequently, all in all, a virtually continuous irradiation is achieved that normally entails less time loss as compared to “classic” gating irradiation procedures. (The same applies analogously in case of the additional and/or alternative use of a dose rate modulation.)

The irradiation of the patient in step 12 is, of course, carried out in such a way that all of the relevant irradiation parameters are also recorded so that, after the irradiation 12, the actually deposited dose can be reconstructed. This knowledge can be incorporated into the irradiation plan for other irradiation cycles that might optionally be carried out in the future (for example, in the next few days). As a result, the quality of the total irradiation carried out by several irradiation cycles can be further improved.

The data that is thus concurrently recorded is stored in a subsequent method step 13 (for example, onto a CD, DVD, hard drive, solid-state memory device, etc.).

During the actual irradiation 12, it can happen that the patient makes irregular movements, for example, if he/she coughs and/or breathes particularly deeply. There are usually no partial irradiation plans for such a fast or especially “broad” movement, precisely because these movements are quite erratic and also occur quite rarely. If such an irregular movement is registered, then the treatment is briefly interrupted by a fast switch-off mechanism. Even though this leads to a slight prolongation of the procedure, it avoids the undesired deposition of a dose into healthy tissue.

FIGS. 4 and 5 once again illustrate the principle for adapting the previously computed irradiation plan (with a safety margin) to deviating boundary conditions at the point in time of the administration of the radiation. Here, the breathing 29 of a patient 22 is used as an example. The principle, however, can also be used for different types of basic parameters.

FIG. 4 as well as FIG. 5 show the course over time along the abscissa 27. The breathing movement 29 of a patient 22 is shown along the ordinates 28. The partial figures a (FIGS. 4a, 5a) each show the movement sequence that was determined within the scope of a preliminary examination (for instance, examination step 3 during the preceding procedure 1). The partial figures b (FIGS. 4b, 5b) each show the situation that arises when the breathing 29 of the patient 22 slows down during the actual irradiation (see irradiation procedure 2). In contrast, the partial figures c (FIGS. 4c, 5c) each show the situation when the patient 22 breathes more rapidly during the actual irradiation 2 than was assumed during the preceding procedure 1.

FIG. 4 shows how the relationship between the irradiation point and the movement phase 30 can be (essentially) maintained by using pause intervals 32. In contrast, FIG. 5 shows how the correlation between the irradiation points 33 and the movement phase 30 can be maintained by using a different dose rate. These two procedures can be used not only on their own but also in combination. As a rule, in actual practice, the correlation can be maintained with a good to excellent approximation when an individual procedure is used as well as in a combination of both procedures.

FIG. 4a shows how the breathing movement 29 can be divided into a plurality of movement intervals 30a, 30b . . . 30f (after the movement interval 30f, the breathing cycle 29 starts again with the movement interval 30a). In drawing up the irradiation plan, it has to be taken into consideration that it is quite probable that the patient 22 will breathe more rapidly (to a certain extent) during the actual irradiation than was determined during the preliminary examination. In order to have sufficient “leeway” here, each individual movement interval 30 is provided in the beginning with an irradiation phase 31 during which a certain number of points is irradiated. For example, in the first movement interval 30a, the points 33 1 to N are irradiated 31a. Each irradiation phase 31 is followed by a pause phase 32. The length of each pause phase 32 is preferably a function of the duration of the corresponding movement phase 30 (for example, a certain percentage such as 10% or 20% thereof), whereby preferably a certain minimum length is provided. No irradiation takes place during the pause phase 32. In the present FIG. 4, for the sake of clarity of the illustration, the interval lengths are not drawn true-to-scale.

Every single movement phase 30 (for example, the movement phase 30a) is followed by another movement phase 30 (for example, the movement phase 30b), which starts once again with an irradiation phase 31 (for example, 31b), and finally, it makes a transition to another pause phase 32 (for example, 32b). During the subsequent interval 30, the irradiation points 33, for instance, with the numbers N+1 to P, are irradiated.

If the patient 22 breathes more rapidly during the actual irradiation 2 than originally “assumed” (see FIGS. 4a to 4c), this faster breathing can be compensated for (up to a certain extent) by shortening the pause phases 32. This shortening of the pause intervals 32 makes it possible to maintain the correlation between the irradiation points 33 and the irradiation phase 30, in spite of the changed breathing 29 of the patient 22. Concretely, for example, during the first movement interval 30a, the irradiation points 33 1 to N continue to be irradiated, whereas during the second movement interval 30b, it is the irradiation points N+1 to P that are irradiated. This adaptation of the irradiation progress to the actual breathing has the major advantage that it is possible to “predict” the radiation deposition into the tissue surrounding the active irradiation point 33 in each case.

Of course, it is also possible that the patient will breath more slowly during the actual irradiation than had been assumed within the scope of the drawing up 1 of the irradiation plan. In order to compensate for such a deviation from the target value, the appertaining pause phases 32 are appropriately lengthened.

FIG. 5 shows how the adaptation between the “assumed” and the “actual” movement speed can be made by changing the irradiation intensity. In this case, if applicable, the use of pause phases 32 can be dispensed with entirely. Instead, the irradiation intensity during faster breathing (FIG. 5c) in comparison to the breathing speed (FIG. 5a) “assumed” within the scope of the irradiation plan is appropriately increased, whereas it is decreased in the case of a slower breathing speed of the patient 22 (see FIG. 5b).

In order to be able to make an adaptation in case of faster breathing (FIG. 5c), the modified maximum dose rate (see FIG. 5a) used for drawing up the irradiation plan has to be smaller than the maximum dose rate of the irradiation system 14 employed. In this context, suitable values have proven to be a 10% to 20% safety margin. Of course, the use of the method illustrated in FIG. 5 presupposes an irradiation system 14 that can be regulated commensurately quickly.

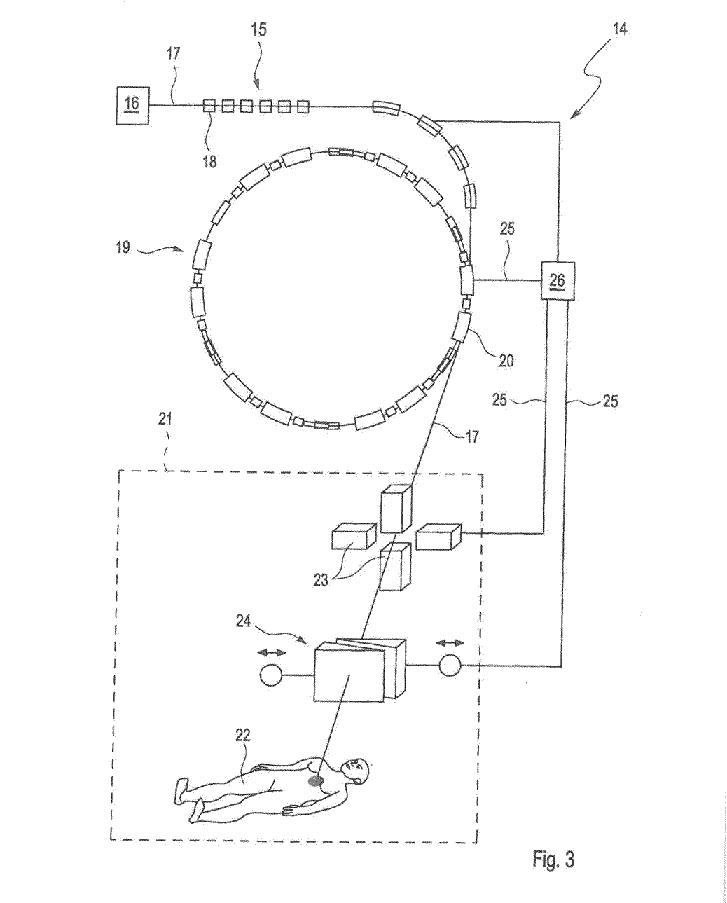

FIG. 3 shows a schematic perspective view of an irradiation system 14 in which the irradiation procedure 2 (optionally also the preceding procedure 1) can be advantageously carried out. The irradiation system 14 is configured as a generally known heavy ion accelerator 15. The heavy ion accelerator 15 has an ion source 16. The ions 17 generated by the ion source 16 are first pre-accelerated in a linear accelerator 18 and shot into a synchrotron ring 19. In the synchrotron ring 19, the ions 17 are further accelerated to the desired high target energy. Once the desired target energy has been reached, the particles located in the synchrotron ring 19 are extracted via an extraction septum 20. A typical duration for an extraction cycle (a so-called particle spill) is between 2 and 10 seconds. The ions 17 extracted via the extraction septum 20 are then conveyed to a treatment room 21 in which the patient 22 is located. In the embodiment presented here, the application of the particles (ions 17) is carried out using a scanning procedure, especially a so-called raster scanning procedure. For this purpose, the pencil-thin particle beam 17 is moved line-wise, column-wise and slice-wise in such a way that a desired three-dimensional volume is irradiated. The particle beam 17 remains in the position in question for a different length of time, depending on how high the individual dose is that is to be administered in each raster point.

The position of the particle beam is deflected in a lateral direction by means of a pair of deflection coils 23. In the longitudinal direction (change of the position of the Bragg peak of the ions 17), a change is made here through an appropriate actuation of a passive energy modulator 24. In this case, in a known manner, two wedge-shaped blocks made of an energy-absorbing material (a different number of wedges is also conceivable) are moved in such a way that the particle beam 17 has to travel a different path through the energy-absorbing material. As a result, a different amount of energy is extracted from the ions 17 that are passing through the passive energy modulator 24. However, it is also possible that a (slower) energy adaptation is made by means of an active adaptation of the synchrotron ring 19. With the currently available synchrotron rings 19, an energy adaptation can be made from one particle spill to the next.

The individual components of the irradiation system 14 are in communication with a control unit 26 via data lines 25. The control unit 26 can consist of a single computer, or of a larger number of computers. The term computer does not necessarily refer to classic computers but can also mean (partially) single-board computers and the like. The control unit 26 carries out the necessary computations (for example, the computations that have to be carried out within the scope of the irradiation procedure 3). Moreover, in the control unit 26, the control signals for the individual components 16, 18, 19, 20, 23, 24 are generated. Furthermore, the control unit 26 receives and appropriately processes the measuring and control signals that are detected by suitable sensors or measuring devices (not shown in FIG. 3).

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B.” Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise.

### LIST OF REFERENCE NUMERALS

1 preceding procedure
2 irradiation procedure
3 examination step
4 digitalization
5 drawing up the initial irradiation plan
6 drawing up the partial irradiation plan
7 storing
8 expanding measures
9 re-optimization step
10 gating division step
11 drawing up the actual irradiation plan
12 irradiation
13 storing
14 irradiation system
15 heavy ion accelerator
16 ion source
17 ions
18 linear accelerator
19 synchrotron ring
20 extraction septum
21 treatment room
22 patient
23 scanner magnets
24 passive energy modulator
25 data lines
26 control unit
27 abscissa
28 ordinate
29 breathing movement
30a, 30b . . . movement interval
31a, 31b . . . irradiation phase
32a, 32b . . . pause phase
33 irradiation position

1-15. (canceled)

16: A method for drawing up an irradiation plan for a radiation-generating device, the irradiation plan including a plurality of irradiation positions that, at least one of partially or at times, correlate with at least one basic parameter that can be present at a point in time of the implementation of the irradiation plan, the method comprising giving greater consideration to correlations with the at least one basic parameter that are expected with greater probability for at least some of the irradiation positions.

17: The method according to claim 16, wherein a division into the irradiation positions and/or a division into groups of irradiation positions is made, at least at times and/or at least partially time-based and/or at least at times and/or at least partially as a function of the basic parameters.

18: The method according to claim 16, wherein a time-variable dose rate of the radiation generated by at least one radiation-generating device is at least one of at times or partially taken into consideration when the irradiation plan is being drawn up, and/or wherein a safety margin is taken into consideration when the irradiation plan is being drawn up.

19: The method according to claim 16, wherein the at least one basic parameter is incorporated into the irradiation plan at least one of at times or partially while making use of data preferably obtained by measurements.

20: The method according to claim 16, characterized in that the irradiation plan is drawn up in several steps, at least one of at times or in certain areas, especially in the form of at least one drawing-up step of the irradiation plan and/or at least one drawing-up step of at least one partial irradiation plan for at least one irradiation position and/or at least one optimization step of the irradiation plan and/or at least one optimization step of at least one partial irradiation plan for at least one irradiation position.

21: The method according to claim 16, wherein the at least one basic parameter represents at least one of:
a movement of at least one irradiation position, especially a periodic movement of at least one irradiation position that occurs at least at times and/or at least partially,
a dose rate of the at least one beam-generating device, especially a time-varying maximum dose rate of the at least one radiation-generating device, or
a time variation of the dose rate generated by the radiation-generating device.

22: The method according to claim 21, wherein the movement of at least one irradiation position, at least at times and/or at least in certain areas, is a translatory movement, a rotatory movement, or movements that shift relative to each other, a compressive movement and/or a stretching movement, and/or a change in terms of density.

23: The method according to claim 16, wherein the at least one radiation-generating device generates particle radiation at least one of at times or partially, especially hadron radiation at least at times and/or at least partially, preferably proton radiation at least at times and/or at least partially, helium ion radiation, carbon ion radiation, neon ion radiation, oxygen ion radiation, pion radiation, meson radiation and/or heavy ion radiation.

24: The method according to claim 16, wherein a correlation is established between at least one irradiation position and at least one basic parameter, especially a correlation between at least one irradiation position and at least one movement phase of the at least one irradiation position.

25: A method for administering a spatially resolved radiation dose in at least one irradiation position using at least one radiation-generating device comprising depositing a dose rate using the at least one radiation-generating device in the at least one irradiation position that is varied, at least one of at times or partially, in correlation with at least one basic parameter that is present at the time of the administration.

26: The method recited in claim 25, wherein the at least one-radiation-generating device includes a particle beam-generating device.

27: The method according to claim 25, wherein the administration of the spatially resolved radiation dose is performed, at least one of at times, partially or in certain areas, while making use of at least one irradiation plan.

28: The method according to claim 27, wherein the irradiation plan is drawn up by giving greater consideration to correlations with the at least one basic parameter that are expected with greater probability for at least some of the irradiation positions.

29: The method according to claim 25, wherein the at least one basic parameter represents at least one of:
a movement of at least one irradiation position, especially a periodic movement of at least one irradiation position that occurs at least at times and/or at least partially,
a dose rate of the at least one beam-generating device, especially a time-varying maximum dose rate of the at least one radiation-generating device, or
a time variation of the dose rate generated by the radiation-generating device.

30: The method according to claim 29, characterized in that the movement of at least one irradiation position, at least at times and/or at least in certain areas, is a translatory movement, a rotatory movement, or movements that shift relative to each other, a compressive movement and/or a stretching movement, and/or a change in terms of density.

31: The method according to claim 25, wherein the at least one radiation-generating device generates particle radiation at least one of at times or partially, especially hadron radiation at least at times and/or at least partially, preferably proton radiation at least at times and/or at least partially, helium ion radiation, carbon ion radiation, neon ion radiation, oxygen ion radiation, pion radiation, meson radiation and/or heavy ion radiation.

32: The method according to claim 25, wherein the deposited dose rate is varied, at least one of at times or partially, by at least one of varying the dose rate generated by the radiation-generating device or discontinuing the dose deposition.

33: The method according to claim 32, wherein a smaller dose rate than the maximum possible dose rate is deposited under standard conditions.

34: A device for drawing up an irradiation plan that is configured and equipped so as to execute the method according to claim 16.

35: A device for actuating at least one radiation-generating device that is configured and equipped so as to execute, at least one of at times or partially, the method according to claim 25.
